(19) United States
(12) Patent Application Publication
(10) Pub. No.: US 2008/0071172 A1
(43) Pub. Date: Mar. 20, 2008

(54) Combined 2D Pulse-Echo Ultrasound And Optoacoustic Signal

(76) Inventors: Abraham Bruck, Haifa (IL); Semion Trebukox, Nazreth (IL); Oma Gayer, Tel-Aviv (IL); Amir Avner, Ramar Yishmy (IL); Moshe Rint, Holon (IL)

Correspondence Address: WOLF, BLOCK, SHORR AND SOLIS-COHEN LLP, 250 PARK AVENUE, 10TH FLOOR, NEW YORK, NY, 10177, US

(21) Appl. No.: 11/615,184

(22) Filed: Dec. 22, 2006

Related U.S. Application Data
(63) Continuation-in-part of application No. PCT/IL06/00066, filed on Jan. 17, 2006.
(60) Provisional application No. 60/753,830, filed on Dec. 23, 2005.

(30) Foreign Application Priority Data
Jan. 20, 2005 (IL) 166408

(51) Int. Cl. A61B 8/00

(52) U.S. Cl. 600/438; 600/439

(57) ABSTRACT

The present invention provides an adjunct apparatus to conventional ultrasound systems and method for combining ultrasound images that reflect pulse echo ultrasound properties together with optoacoustic properties of tissues and body fluids as a function of spatial location. The present invention in another aspect consists of the addition of a new operating mode to ultrasound imaging systems.

This present application claims the benefit of earlier IL patent application S.N. 166408 filed on Jan. 20, 2005 by Bruck Abraham et al. and entitled “Combined 2D Pulse-Echo Ultrasound and Optoacoustic signal for Glaucoma Treatment” and is a continuation-in-part of U.S. provisional patent application Ser. No. 60/753830 filed on the 28^{th} of Dec. 2005 entitled “Operating mode for Ultrasound imaging systems.

### FIELD OF THE INVENTION

The present invention relates to ultrasound imaging combined with optoacoustic signal. More particularly, the present invention relates to utilization of combined 2D pulse-echo ultrasound and optoacoustic signal for medical needs.

### BACKGROUND OF THE INVENTION

Ultrasound imaging of small part structures including breast, thyroid, prostate; ophthalmic structures; cardiac structures; the peripheral vascular systems; the fetus and uterus; abdominal organs such as the liver, kidneys, and gall bladder; skin structures is a known medical imaging technique. Ultrasound imaging is based on transmission of short ultrasound pulses along a definite direction and receiving the ultrasound echoes from the different tissue interfaces along the propagation direction of the ultrasound pulse. The arrival time of the echoes determine the distance of the echo source from the ultrasound transmitter/receiver. A complete image can be reconstructed by varying the direction of the ultrasound beam and recording the echo intensities as a function of direction and distance. The beam direction can be varied by mechanically moving a single transmit/receive ultrasound transducer, or by electronic means using an array of transducers. Usually the same transducer is used for transmitting and for receiving. This type of image displays tissue interfaces with intensities proportional to the reflection coefficients of these interfaces providing anatomic information.

Optoacoustic imaging of ophthalmic; brain; peripheral vascular; small parts including breast, thyroid, prostate structures is also a known method. The optoacoustic imaging is based on transmitting short pulses of electro-magnetic radiation, for example light using a laser that can be a narrow beam along a definite direction, or a spread out beam illustrating a selected volume. The laser beam excites ultrasound in the tissue that now becomes an ultrasound source. The ultrasound is detected by an ultrasound receiver, or array of receivers, to produce a complete image, or a signal distribution along a single laser beam direction. This type of image represents the characteristic of the laser light absorption (function of wave-length), the elasticity, and the thermal properties of the tissue.

Examples of using various forms of electro-magnetic radiation in optoacoustic imaging are disclosed in several patent disclosures such as:
1. U.S. Pat. No. 4,385,634 “Radiation induced thermoacoustic imaging” filed in 1981 by Bowen.
2. U.S. Pat. No. 6,652,459 “Ophthalmic uses of lasers” filed in 2001 by Payne et al. This patent teaches a method for analyzing and therapy of the eye utilizing laser-induced ultrasound.
3. U.S. Pat. No. 5,840,023 “Optoacoustic imaging for medical diagnosis” filed in 1996 by Oraevsky et al.
4. European patent EP 920277 (application 97904228.0) European version of 3.
5. U.S. Pat. No. 6,309,352 “Real time optoacoustic monitoring of changes in tissue properties” filed in 1998 by Oraevsky et al.
6. German patent DE 4400674A1 Niesner at al. filed 1994
7. European patent 0282234A1 Dowling filed in 1988
8. Photoacoustic Ultrasound Theory—Robert A. Kruger—SPIE Vol. 2134A,
9. Laser based optoacoustic optoacoustic imaging in biological tissues—Oraevsky at al—SPIE Vol. 2134A.

Combination of ultrasound echo intensity image with other echo properties such as tissue motion image (Color Flow Imaging), using the Doppler effect to analyze the ultrasound echo, is a known method for imaging of blood flow and tissue motion. The excitation source for both is the same ultrasound source, and the ultrasound echo is analyzed.

In the present invention, the two methods are combined while the optoacousticaly generated ultrasound data is overlaid on the pulse echo ultrasound image, in real time. The method produces a combined image that reflects the pulse echo ultrasound properties together with the optoacoustic properties of the tissue as a function of spatial location.

In most recent years, the need for such combination was expressed by researchers in the industry and examples can be viewed at:
1. Emilianov et al.—“Combined ultrasound, optoacoustic, and elasticity imaging” Proceedings SPIE Vol. 5320, (2004).
2. Niedeerhouser et al.—“Combined ultrasound and optoacoustic system for real time, high contrast, vascular imaging” IEEE transactions on medical imaging vol. 24 no. 4, (2005).

However, to the best of the inventors knowledge, there is no description and no reference in the prior art of how to combine the two methods in order to achieve the results that are needed for real time imaging.

Regulation of the intraocular pressure is an accepted treatment for glaucoma. One of the established methods is transscleral laser cyclophotocoagulation of small parts of the ciliary body. The main problem with the available method is the exact localization of the relevant target and the possibility of following up the outcome of the procedure in real time. Localization of an opthalmic operation is disclosed in DE patent no. 19916653 “Laser cyclo-photocoagulation for treatment of the ciliary body in cases of intractable glaucoma uses opto-acoustic tissue differentiation so that tissue type is more accurately determined and an appropriate dose applied” by Bruder et al., which was published in 2000. However, the procedure that is performed is a pre-operational procedure in which optical characteristics are established so as to plan the operation.

According to one aspect of the present invention, an external addition to conventional ultrasound systems is described enabling the combination of 2D pulse-echo ultrasound imaging, which is essential to understand the anatomy of tissue structures, with optoacoustic (thermoacoustic) imaging which provides information regarding optical and thermal properties of tissue, adding a new diagnostic capability to conventional ultrasound systems.

In another aspect of the present invention, integration of a new operating mode into pulse echo ultrasound systems is provided. The new mode enables real-time combination of pulse-echo imaging with thermoacoustic (optoacoustic) imaging and displays both images as one combined image.

According to a further aspect the present invention, the apparatus provides a device for the measurement of the concentration of substances in body fluids in vivo.

### SUMMARY OF THE INVENTION

It is an object of the present invention to provide an apparatus and method that can be added to conventional pulse-echo ultrasound system, for combining image that reflects the pulse echo ultrasound properties and optoacoustic (thermoacoustic) image that reflects optical and thermal properties of tissue.

It is another object of the present invention to provide an apparatus that overlays ultrasound signals generated by electro-magnetic radiation such as laser beam through the optoacoustic (thermoacoustic) effects on top of a standard, 2D real time ultrasound image. The overlaid optoacoustic image provides information regarding the concentration of substances in body fluids.

It is yet another object of the present invention to provide a mode of operation as an integral part of ultrasound imaging system enabling combination of pulse-echo ultrasound imaging with optoacoustic imaging.

In addition, it is provided in accordance with another preferred embodiment of the present invention an apparatus for guiding a laser beam focused to a predetermined position. Perform treatment and follow up treatment at the predetermined position, the apparatus comprising a pulse-echo ultrasound system adapted to receive and process the optoacousticaly generated ultrasound signals, either by using an attachment which enables the excitation, acquisition, processing of optoacoustic data and displaying a combined image, or by using an ultrasound system which has an integral mode of optoacoustic imaging.

It is therefore provided in accordance with a preferred embodiment of the present invention an apparatus adjunct to conventional pulse-echo ultrasound systems adapted to add the capability of combining pulse-echo ultrasound data with optoacoustic (thermoacoustic) data and display a combined image, the apparatus comprising:
pulsed electro-magnetic source adapted to generate radiation;
hardware/software for acquisition, processing and optoacoustic image generation;
pulse-echo ultrasound image and optoacoustic image merger;
signal and control lines connecting the conventional pulse-echo ultrasound system with the apparatus.

Furthermore and in accordance with another preferred embodiment of the present invention, said electromagnetic source is selected from a group of sources such as laser, microwave, or radio frequency.

Furthermore and in accordance with another preferred embodiment of the present invention, the apparatus is adapted to perform measurement of concentration of substances in body fluids and generating an optoacoustic image combined with a pulse-echo ultrasound image as a function of said concentration.

Furthermore and in accordance with another preferred embodiment of the present invention, the apparatus comprises:
attachment fixed to a probe of the conventional pulse-echo ultrasound system,
an optical fiber provided to said attachment wherein said optical fiber allows a laser beam to be directed to a predetermined position relative to said probe, and wherein said laser beam generates radiation adapted to be directed to a predetermined position;
an array of ultrasound receivers including at least three receivers provided in said attachment, wherein said array of ultrasound receivers is adapted to sense the signal generated by said radiation;
hardware/software adapted to acquire, and process an optoacoustic image, generate, control, and merge pulse-echo ultrasound and optoacoustic images;
signal and control lines connecting the conventional pulse-echo ultrasound system with the attachment.

Furthermore and in accordance with another preferred embodiment of the present invention, the apparatus comprises:
an attachment fixed to a probe of the conventional pulse-echo ultrasound,
an optical fiber adapted to allow a laser beam to be directed to a predetermined position relative to said probe, wherein said laser beam generates radiation that is adapted to be directed to the predetermined position,
hardware/software for acquire, process, generate and control optoacoustic image, merge pulse-echo ultrasound and optoacoustic image;
signal and control lines connecting the conventional pulse-echo ultrasound system with the attachment;
a switching circuit adapted to switch output of said probe between the pulse-echo ultrasound system and said hardware/software for the optoacoustic signals.

Furthermore and in accordance with another preferred embodiment of the present invention, the laser beam is focused to a predetermined position and said optoacoustic signal is overlaid over a real-time 2D ultrasound image so as to establish a target for treatment and treatment monitoring of the tissue at the predetermined target position.

Furthermore and in accordance with another preferred embodiment of the present invention, the predetermined position is a ciliary body in the eye.

Furthermore and in accordance with another preferred embodiment of the present invention, said radiation imparts power for treatment.

Furthermore and in accordance with another preferred embodiment of the present invention, a standoff is provided to said probe.

In addition and in accordance with yet another preferred embodiment of the present invention, it is further provided an ultrasound imaging apparatus supporting pulse-echo ultrasound modes of operation as well as optoacoustic, (thermoacoustic) ultrasound mode of operation and displaying simultaneously the mode relevant images overlaid one on top of the other on a combined image; the system comprising:
conventional pulse-echo ultrasound probe;
pulsed electro-magnetic source generating radiation;
control lines connecting said pulsed electro-magnetic source and said conventional pulse-echo ultrasound probe.

Furthermore and in accordance with another preferred embodiment of the present invention, said source is selected from a group of radiation sources such as laser, microwave, or radio frequency.

Furthermore and in accordance with another preferred embodiment of the present invention, the apparatus adapted to perform measurement of the concentration of substances in body fluids and generating an optoacoustic image combined with a pulse-echo ultrasound image, as a function of said concentration.

Furthermore and in accordance with another preferred embodiment of the present invention, further comprising an attachment fixed to the conventional pulse-echo ultrasound probe that includes an optical fiber allowing a laser beam to be directed to a predetermined position relative to said ultrasound probe, wherein said laser beam generates radiation that is adapted to be directed to the predetermined position.

Furthermore and in accordance with another preferred embodiment of the present invention, the laser beam is focused to the predetermined position and the optoacoustic signal is overlaid over a real-time 2D ultrasound image so as to establish a target for treatment and treatment monitoring of the tissue at the predetermined target position.

Furthermore and in accordance with another preferred embodiment of the present invention, said radiation imparts power for treatment.

Furthermore and in accordance with another preferred embodiment of the present invention, said radiation is delivered to the body surface through an optical fiber that is an integral part of said pulse-echo ultrasound probe.

Furthermore and in accordance with another preferred embodiment of the present invention, the predetermined position is a ciliary body in the eye.

Furthermore and in accordance with another preferred embodiment of the present invention, the operation sequence comprising:
performing a first sequence of a least 1 pulse-echo along a predetermined axial direction;
performing a second sequence of at least one electromagnetic excitation along said axial direction;
performing said first sequence and said second sequence along multitude axial directions;
constructing a final image from signals received from said multitude axial directions.

Furthermore and in accordance with another preferred embodiment of the present invention, the operation sequence comprises:
generating one complete pulse-echo image frame;
generating a complete electromagnetically excited image frame to produce a final image; and
combining final image from said pulse-echo images frame and excited image frame.

### BRIEF DESCRIPTION OF THE FIGURES

In order to better understand the present invention and appreciate its practical applications, the following Figures are attached and reference herein. Like components are denoted by like reference numerals.

It should be noted that the figures are given as examples and preferred embodiments only and in no way limit the scope of the present invention as defined in the appending Description and Claims.

FIG. 1a illustrates a side view of an ultrasound probe provided with an add-on attachment in accordance with a preferred embodiment of the present invention.

FIG. 1b illustrates a block diagram of the apparatus shown in FIG. 1a.

FIG. 2a illustrates a side view of a combined pulse-echo ultrasound and optoacoustic signal having a shared probe in accordance with a preferred embodiment of the present invention.

FIG. 2b illustrates a block diagram of the apparatus shown in FIG. 2a.

FIG. 3a illustrates a side view of an ultrasound probe combined with optoacoustic signal in accordance with anther preferred embodiment of the present invention.

FIG. 3b illustrates a block diagram of the apparatus shown in FIG. 3a.

FIG. 3c illustrates a block diagram of an ultrasound probe combined with optoacoustic signal in accordance with yet another preferred embodiment of the present invention.

FIG. 4 illustrates an implementation of a hardware/software part of an add-on device to a conventional ultrasound system in accordance with a preferred embodiment of the present invention operating with mechanical probes as well as with electronic arrays.

FIG. 6a illustrates an add-on attachment to ultrasound systems provided with new mode of operation supporting optoacoustic imaging in accordance with a preferred embodiment of the present invention, wherein the laser fiber is attached to the ultrasound probe.

FIG. 6b illustrates an add-on attachment to ultrasound systems provided with new mode of operation supporting optoacoustic imaging in accordance with another preferred embodiment of the present invention, wherein the laser fiber moves freely on the body.

FIG. 7 illustrates an exemplary solution for an ultrasound system having a mode of operation providing for optoacoustic imaging together with pulse-echo 2D pulse echo ultrasound imaging in accordance with a preferred embodiment of the present invention.

FIG. 8 illustrates an exemplary solution for an ultrasound system having a mode of operation providing for optoacoustic imaging together with pulse-echo 2D pulse echo ultrasound imaging in accordance with another preferred embodiment of the present invention.

### DETAILED DESCRIPTION OF THE INVENTION AND THE FIGURES

The present invention provides a novel and unique adjunct apparatus and method to be added to conventional ultrasound system providing in real time combined anatomic and functional (optoacoustic) image. In according to another aspect of the present invention, the innovation in another embodiment of the present invention consists of inclusion of a new operating mode into ultrasound imaging systems. The new mode will enable real-time combination of pulse echo ultrasound imaging with thermoacoustic (optoacoustic) imaging and display both images as one combined image.

According to one aspect of the present invention, attachment that can be fitted to a multitude of standard real time ultrasound imaging probes (transducers) is provided. The attachment can include:
A fiber that delivers the laser beam;
An ultrasound sensor array consisting of at least three, but not limited to, sensors for receiving the optoacoustic signals. The sensors are located at fixed positions relative to the ultrasound probe.

Reference is now made to FIG. 1a illustrating a side view of an ultrasound probe provided with an add-on attachment in accordance with a preferred embodiment of the present invention. An attachment 3 is attached to an ultrasound (ULS) probe 2 that can be a standard ultrasound probe. The probe can be a mechanical scanner as shown in this embodiment or an electronic array as illustrated in other embodiments as will be shown herein after. Attachment 3 comprises a fiber 6 adapted to guide a laser beam having an illumination range substantially defined as an area 37 between two doted lines.

Optionally, a lens or a lens assembly is provided on an expected path of the laser beam that is propagating of the fiber. The lens is adapted to be variable, or replaceable in accordance with the specific application.

Attachment 3, which is adapted to be adjacent to a bodily area that is imaged to receive a standard ULS radiation of an image area 36, is further provided with opto-acoustic signal receivers that optionally may be ultrasound sensor array 32 consisting of wide band omni directional (for example 5 to 30 MHz) ultrasound receivers.

A space 35 between ULS probe 2 and the examined area 36 is filed with water 35 while a sealing membrane that is transparent to ultrasound and laser light 38, provides a boundary between water 35 and the bodily area.

In an application in which the apparatus of the present invention is used in ophthalmic operation, the treated area in the ciliary body of the eye is treated with laser beam and produces optoacoustic signal that is backwardly directed towards the attachment of the present invention and is received by opto-acoustic signal receivers. The laser beam is generating opto-acoustic signal, or alternatively can generate power for treatment (the localization laser source and the treatment laser source can be different lasers coupled into the same fiber).

The combined apparatus is provided with a hardware and software having the following main features:
1. Determination and display of the spatial position and physical characteristics of the optoacoustic signals received by the sensor array.
2. Import the real time ultrasound image from the conventional ultrasound system and following a geometrical calibration, display it together with the optoacoustic data.
3. Control the integrated system operation, such as the timing of the activation of the localizing laser and the treatment laser, if two separate lasers used, or the activation of the different operation modes of a single laser.

Following is an example of a method providing combined imaging in accordance with a preferred embodiment of the present invention:

An assembly of a standard ultrasound probe (preferably a high frequency linear array probe) and a laser fiber is used to image the target site through a water-path, according to the procedure as follows:
1. A standard 2D ultrasound image is obtained.
2. The suspicious area is brought into the region marked as the laser target area.
3. The laser is activated to obtain a colored vasculature image overlaid on top of the 2D ultrasound image. The shades of the color are designed to reflect the concentration of substances in body fluids, i.e. the level of blood oxygenation as measured by the optoacoustic modality.

Following is an example of a method of treatment in accordance with a preferred embodiment of the present invention:
1. The target of interest, for example the eye, is scanned with a standard ultrasound system and probe that comprises the attachment shown herein in FIG. 1 and the expected position of the laser focus is overlaid on top of the real-time ultrasound image.
2. The laser focus is brought to the anatomical site of interest by manipulating the ultrasound probe and attachment assembly.
3. During the scan, a pulsed laser is periodically activated to produce the optoacoustic signal from the anatomical site of interest; the spatial location of the optoacoustic signal is determined by the ultrasound sensors and displayed as an overlay on top of the real time ultrasound image. The displayed optoacoustic signal should coincide with the laser focus position. Slight misalignments are corrected by further manipulating the ultrasound probe and attachment assembly.
4. At that time, a treatment laser signal is activated for preset time duration.
5. At the end of that preset time duration, the pulsed laser is activated to observe changes in the optoacoustic signal as a result of the treatment. It is assumed that the variations reflect the effect of the treatment thus they enable the operator to decide regarding the termination, or repetition of the treatment of the selected anatomical site.

Optionally, the laser producing the optoacoustic signal and the treatment laser can be two separate lasers connected to the same fiber, or it can be a single laser activated at two different modes of operation.

Reference is now made to FIG. 1b illustrating a block diagram of the apparatus shown in FIG. 1a. The innovation of the present invention is exhibited in an attachment hardware & software 33 that acquires information from attachment 3 and receives also information from ULS probe 2 through conventional ultrasound system 7. Attachment hardware & software has main functions as follows:
The software receives ultrasound image data from conventional ULS system 7 that is provided in the apparatus as well as opto-acoustic data that is received from attachment assembly 3 that is provided adjacent and on ULS probe 2. Laser or lasers 4 transmit the laser beam through attachment assembly 3 by fiber 6, as shown herein before. The hardware controls also laser or lasers 4. Both data information is overlaid so as to provide the position of the targets responding, via generation of ultrasound, to the laser beam excitation, over the ultrasound image.
Supply triggering for the laser sources.
Acquire and process the Opto-Acoustic signals to localize their origin and overlay them on the ultrasound image.
Display the amplitude and other relevant properties of the relevant Opto-Acoustic signal on a PC or laptop having a display monitor as will be shown herein after.

In accordance with another aspect of the present invention, the combined apparatus can be integrally built.

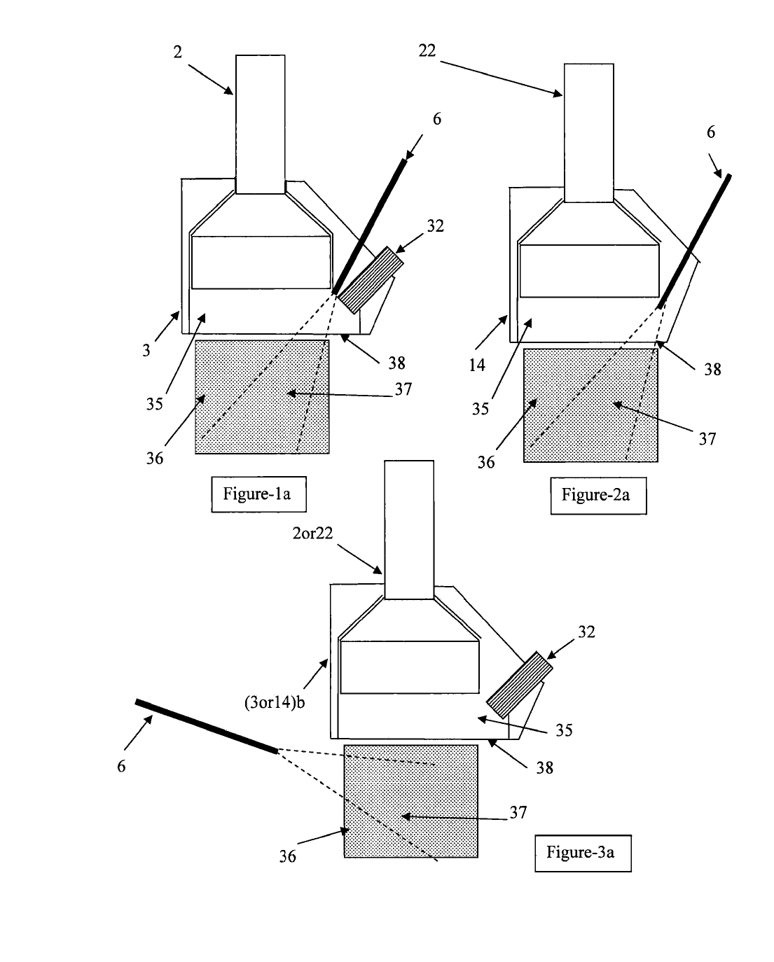

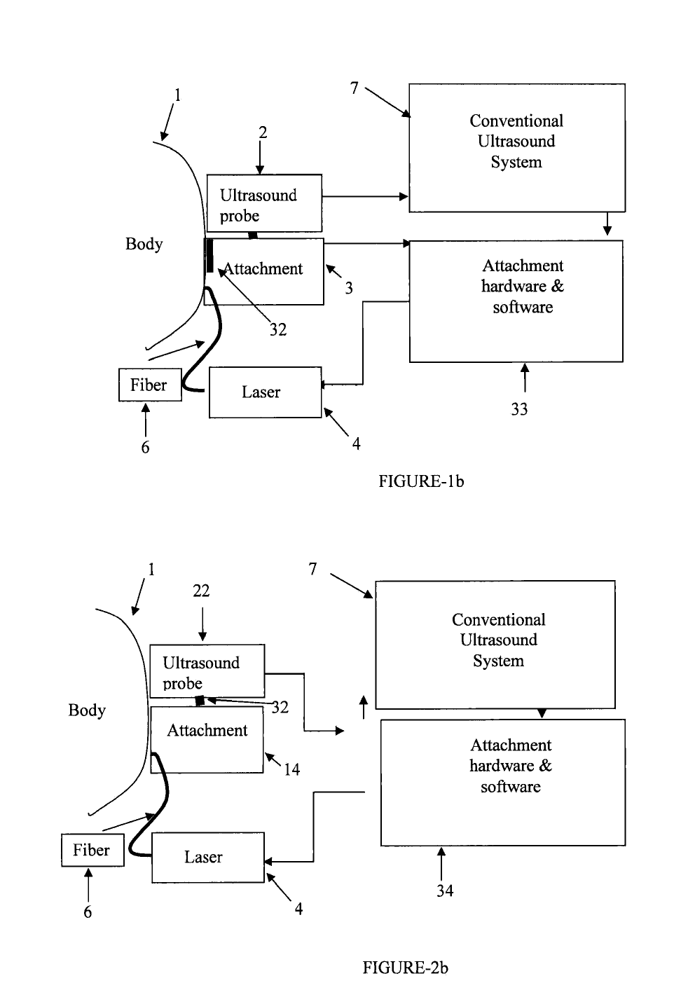

Reference is now made to FIGS. 2a and 2b illustrating a side view and block diagram of a combined pulse-echo ultrasound and optoacoustic signal having a shared probe in accordance with a preferred embodiment of the present invention. An integral assembly of an ultrasound imaging probe 22 and a laser fiber 6 that is attached in attachment 14 is provided. The assembly is shown in a cross sectional side view in FIG. 2a and in block diagram in FIG. 2b. Ultrasound probe 22 is used for the standard, pulse echo, ultrasound imaging and for the acquisition of the ultrasound signals generated by the optoacoustic effect. In this case, the probe consists of an electronic array 22. The electronic array 22 is connected to the attachment hardware/software, which in turn is connected to the probe input of the conventional ultrasound system.

The ultrasound array can be a phased linear array, or a phased linear convex array, or sector phased array, consisting of a multitude of elements.

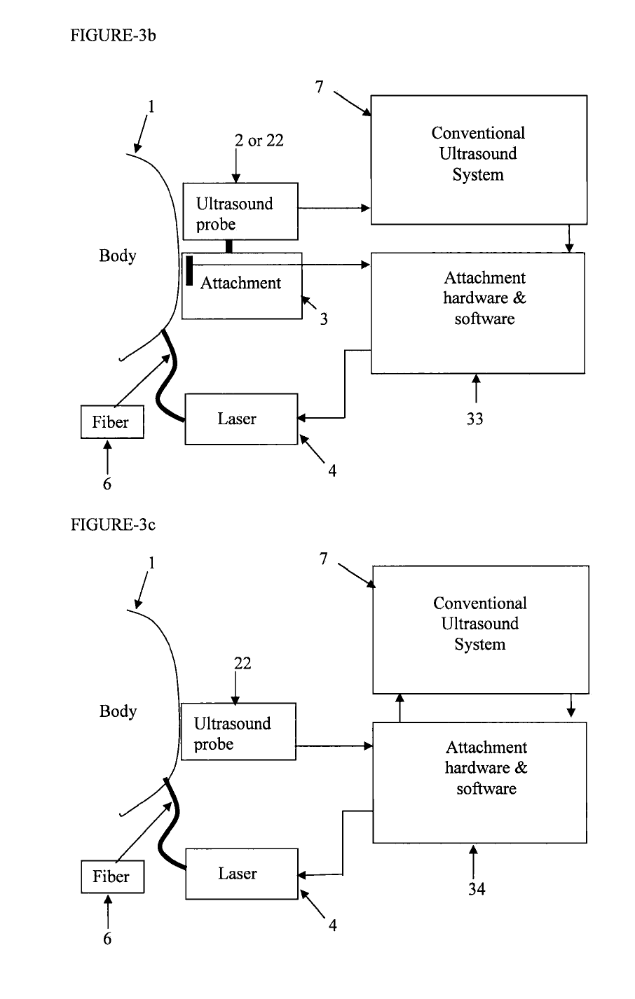

Reference is now made to FIGS. 3a illustrating a side view of an ultrasound probe combined with optoacoustic signal in accordance with another preferred embodiment of the present invention, and FIGS. 3b and c illustrating block diagrams of two different implementations of the apparatus shown in FIG. 3a. Basically, the embodiment is similar to the embodiment shown in FIG. 1; however, laser fiber 6 can be detached from attachment 3 and can be directed in different direction towards the area 36 that is imaged by the ultrasound. Area 37 that is bounded between the doted lines can be changed by moving fiber 6 to another direction. It should be noted that the fiber can be directed towards the region of interest from any direction, from outside the body, or through body cavities, or through catheter.

As shown in FIGS. 3b and c, the receivers can be arranged in the attachment to the ULS or integrated within the probe.

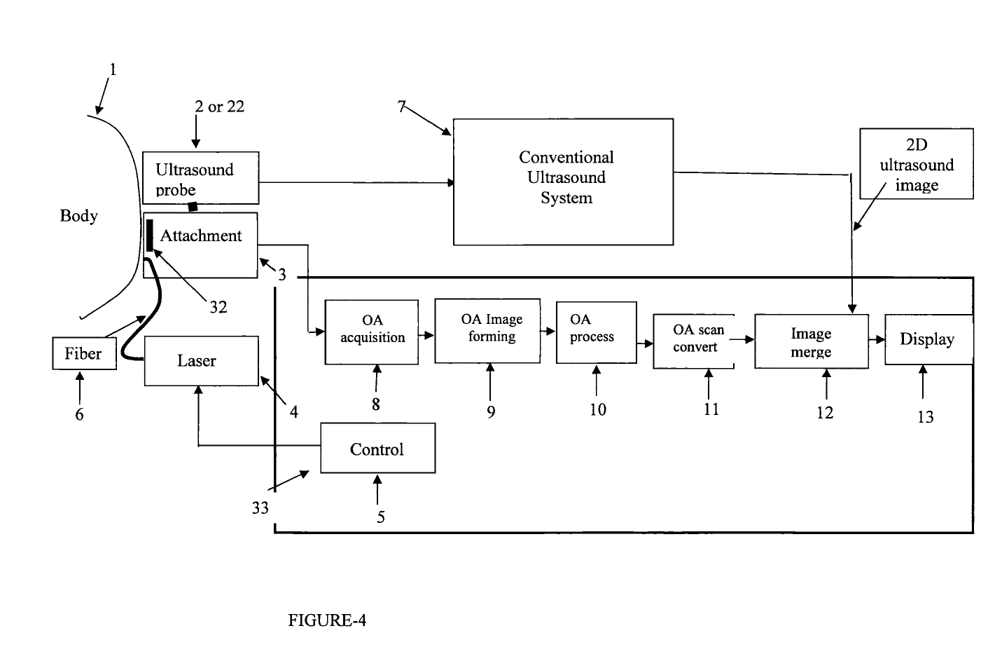

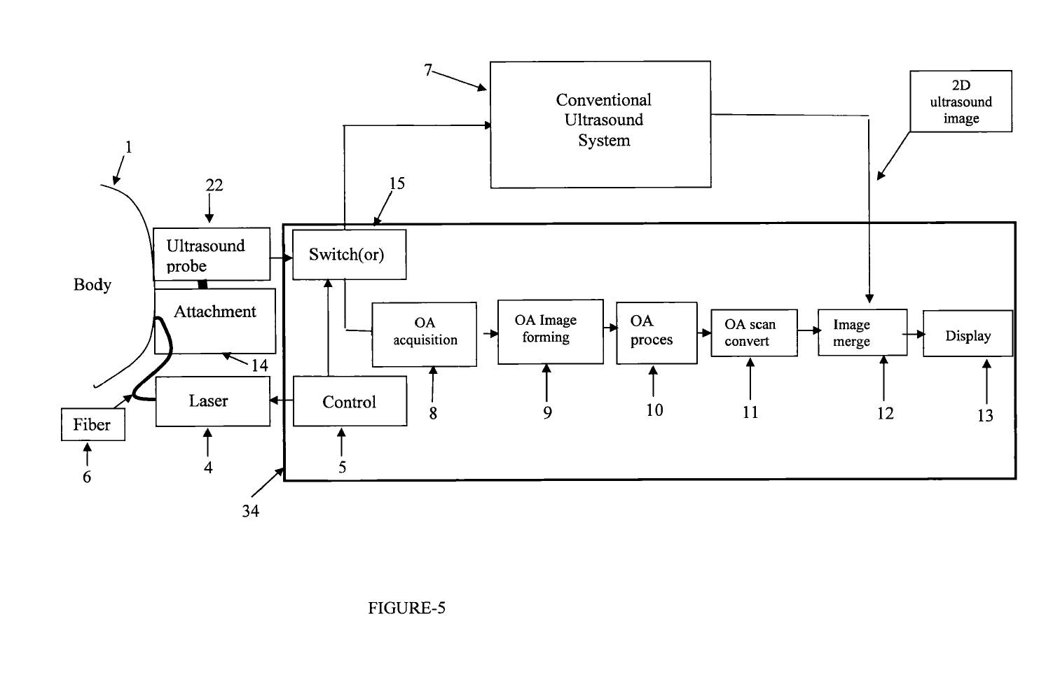

Reference is now made to FIG. 4 illustrating an implementation of a hardware/software part of an add-on device to a conventional ultrasound system in accordance with a preferred embodiment of the present invention operating with mechanical probes as well as with electronic arrays. This exemplary implementation can be applied in the embodiment shown in FIG. 1a as an example wherein the attachment comprises receivers array.

The signals from receiver array 32 are connected to hardware & software processor 33 as shown in FIG. 1b. Processor 33 comprises acquisition 8 by which the optoacoustic signals are acquired, an image former 9 that forms an image out of the signals, a processor 10, scan converter 11 adapted to provide a two dimensional image of the optoacoustic sources. The optoacoustic image is merged by a merger 12 with an image received from conventional ultrasound system 7. A 2D ultrasound image is transferred from conventional ULS system 7 to merger 12. The combined image is displayed on monitor 13 that can be any display. The laser sequence is controlled by controller 5.

Reference is now made to FIG. 5 illustrating an add-on attachment to ultrasound systems provided with new mode of operation supporting optoacoustic imaging in accordance with a preferred embodiment of the present invention, wherein the laser fiber is attached to the ultrasound probe. In the integrated version of the apparatus as shown in FIG. 2a, the optoacoustic signal receivers are integrated within ultrasound probe 22 which is an electronic array. Probe 22 is connected to hardware & software processor 34. Processor 34 comprises a switch 15 switching the probe elements between conventional ultrasound system 7 and the optoacoustic data acquisition and processing unit. The switch is controlled by controller 5. The other components of processor 34 comprises similarly to previous 33, acquisition 8, image former 9, processor 10, scan converter 11 adapted to provide a two dimensional image of the optoacostic sources. The optoacoustic image is merged by merger 12 with the image received from conventional ultrasound system 7. The combined image is displayed on monitor 13. The laser sequence is controlled by controller 5.

In accordance with another aspect of the present invention, the apparatus consists of the addition of a new operating mode to ultrasound imaging systems. The new mode will enable real-time combination of pulse echo ultrasound imaging with thermoacoustic (optoacoustic) imaging and display both images as one combined image.

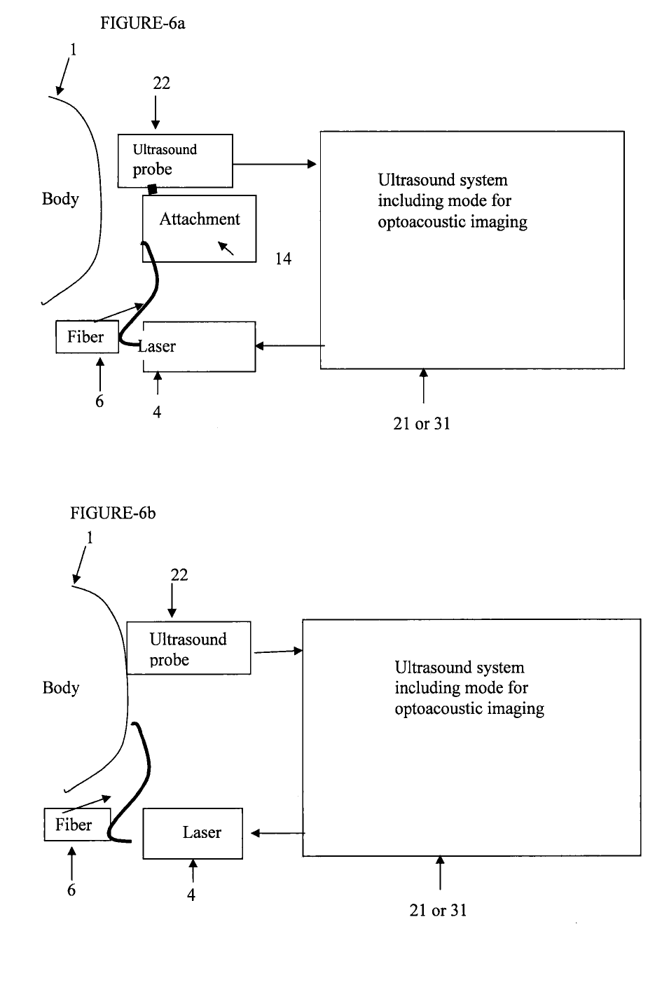

Reference is now made to FIGS. 6a and b illustrating add-on attachments to ultrasound systems provided with new mode of operation supporting optoacoustic imaging in accordance with preferred embodiments of the present invention, wherein the laser fiber is attached to the ultrasound probe and moved freely on the body, respectively.

In a pulse echo system, an ultrasound pulse is transmitted along a predetermined direction and the ultrasound echoes are received as a function of time, using the ultrasound propagation velocity, the time is translated to distance along the predetermined direction:
2d=vt

The factor 2 accounts for the fact that the transmitted ultrasound and the reflected ultrasound propagate at the same velocity. A 2D image is obtained by repeating the procedure along a set of directions generating a 2D area and displaying the relevant echo intensity as brightness, (B mode).

For the thermoacoustic excitation, (optical, microwave, etc.), the generation of the ultrasound is by the electromagnetic radiation having a propagation velocity much higher then that of the ultrasound (actually relative to the ultrasound velocity it can be assumed as infinite).

To enable the imaging of the thermoacousticaly excited ultrasound, in the new operating mode, the transmission of the ultrasound is disabled, the time dependent receiving is correlated with the timing of the external excitation pulse, the calculation of the distance along the receiver direction should now be:
d=vt

### IMPLEMENTATION EXAMPLE-1

Receiving along each direction, at least twice; once in pulse echo method and once in thermoacoustic method as described above, generating relevant images. Both images are displayed one over the other at the correct geometrical locations. Displaying the thermoacoustic image in a different color that the ULS image will show the thermoacoustic properties on top of the pulse echo properties.

### IMPLEMENTATION EXAMPLE-2

Generate a complete 2D image with the pulse echo method, then generate a second 2D image with the thermoacoustic method, adjust scaling and display the images one on top of the other.

It should be mentioned that all the consideration above can be applied also for synthetic aperture imaging methods; this is without limiting the scope of the present invention.

The ULS system that includes the mode for optoacoustic imaging 21 or 31, as described herein before is electronically connected to ULS probe 22 as well as to laser 4 as shown in FIGS. 6a and b in both the add-on attachment through which the laser beam illuminates and an attachment in which the laser is freely disposed on the body.

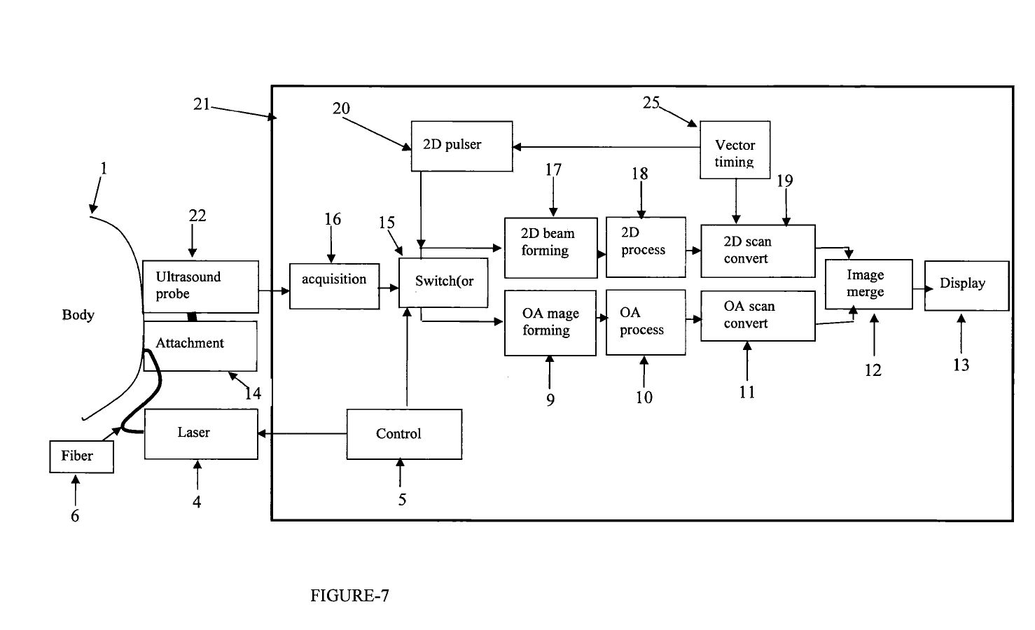

Reference is now made to FIG. 7 illustrating an exemplary solution for an ultrasound system having a mode of operation providing for optoacoustic imaging together with pulse-echo 2D pulse echo ultrasound imaging in accordance with a preferred embodiment of the present invention. Ultrasound system 21 comprises the following main components:

The electronic array of ULS probe 22 is connected through an acquisition unit 16 to a switch 15 switching between 2D data processing that comprises 2D beam former 17, 2D process 18, 2D scan converter 19, and the optoacoustic processing that comprises optoacoustic image former 9, optoacoustic processing 10, and optoacoustic scan converter 11. The switch is controlled by a controller 5 that controls also the laser sequence. The optoacoustic image and the 2D pulse echo ultrasound images are merged by a merger 12 and the combined image is displayed on monitor 13.

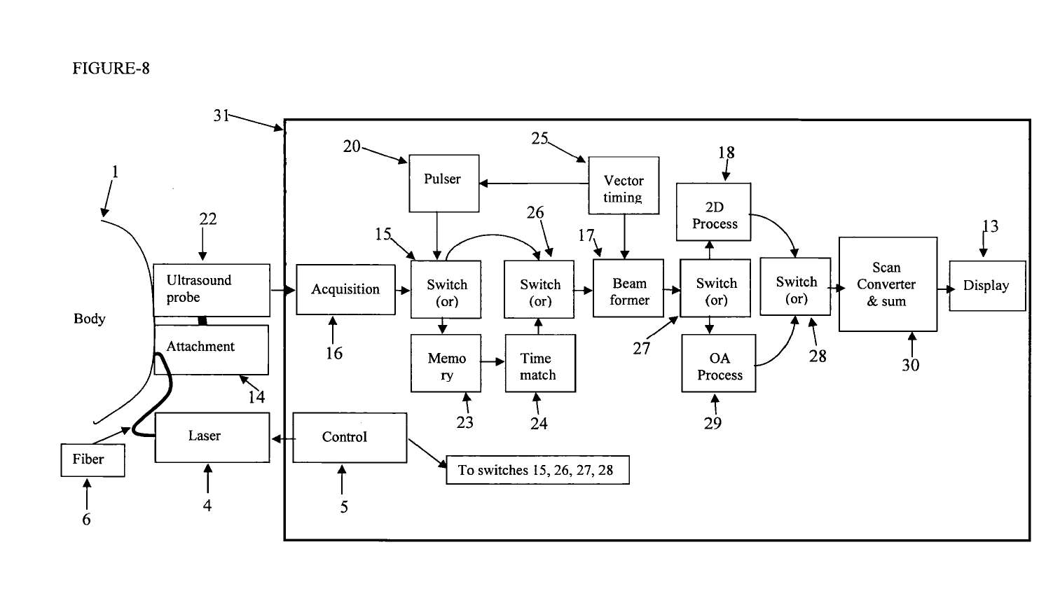

Reference is now made to FIG. 8 illustrating an exemplary solution for an ultrasound system having a mode of operation providing for optoacoustic imaging together with pulse-echo 2D pulse echo ultrasound imaging in accordance with another preferred embodiment of the present invention. In this second example of the main components of ultrasound system 31, which is shown in FIG. 6b, the system comprises the new mode of operation arranged in a different manner than in the embodiment before.

The probe elements 22 are electronically connected through an acquisition unit 16 to a first switch 15 and a second switch 26 switching between 2D bema former 17 and a memory 23. Since each laser pulse generates optoacoustic signal in the whole illuminated region, the memory is required to store the optoacoustic signal received by each element of the receiver array in the attachment. Beam former 17 receives data either directly from the probe that scans the body, or from the memory component 23 by scanning it through a time matching circuit 24. The memory contains the optoacoustic data, the time matching circuit takes care of the fact that the time of flight for pulse echo is twice the time of flight for optoacoustics. The output of beam former 17 is switched by a third switch 27 between 2D processing 18 and optoacoustic processing 29. The output of the processing is directed through switch 28 to scan converter 30 combining the two image sources and displaying them on monitor 13. The laser sequence is controlled by controller 5.

It should be noticed that any other form of arrangement that implement the combined imaging can be used in the ULS system without limiting the scope of the present invention.

It should be clear that the description of the embodiments and attached Figures set forth in this specification serves only for a better understanding of the invention, without limiting its scope as covered by the following Claims.

It should also be clear that a person skilled in the art, after reading the present specification can make adjustments or amendments to the attached Figures and above described embodiments that would still be covered by the following Claims.

1. An apparatus adjunct to conventional pulse-echo ultrasound systems adapted to add the capability of combining pulse-echo ultrasound data with optoacoustic (thermoacoustic) data and display a combined image, the apparatus comprising:
pulsed electro-magnetic source adapted to generate radiation;
hardware/software for acquisition, processing and optoacoustic image generation;
pulse-echo ultrasound image and optoacoustic image merger;
signal and control lines connecting the conventional pulse-echo ultrasound system with the apparatus.

2. The apparatus as claimed in claim 1, wherein said electromagnetic source is selected from a group of sources such as laser, microwave, or radio frequency.

3. The apparatus as claimed in claim 1, wherein the apparatus is adapted to perform measurement of concentration of substances in body fluids and generating an optoacoustic image as a function of said concentration, combined with a pulse-echo ultrasound image.

4. The apparatus as claimed in claim 1, wherein the apparatus comprises:
attachment fixed to a probe of the conventional pulse-echo ultrasound system,
an optical fiber provided to said attachment wherein said optical fiber allows a laser beam to be directed to a predetermined position relative to said probe, and wherein said laser beam generates radiation adapted to be directed to a predetermined position;
an array of ultrasound receivers including at least three receivers provided in said attachment, wherein said array of ultrasound receivers is adapted to sense the signal generated by said radiation;
hardware/software adapted to acquire, and process an optoacoustic image, generate, control, and merge pulse-echo ultrasound and optoacoustic image;
signal and control lines connecting the conventional pulse-echo ultrasound system with the attachment.

5. The apparatus as claimed in claim 1, wherein the apparatus comprises:
an attachment fixed to a probe of the conventional pulse-echo ultrasound,
an optical fiber adapted to allow a laser beam to be directed to a predetermined position relative to said probe, wherein said laser beam generates radiation that is adapted to be directed to the predetermined position,
hardware/software to acquire, process, generate and control optoacoustic image, merge pulse-echo ultrasound and optoacoustic image;
signal and control lines connecting the conventional pulse-echo ultrasound system with the attachment;
a switching circuit adapted to switch output of said probe between the pulse-echo ultrasound system and said hardware/software for the optoacoustic signals.

6. The apparatus as claimed in claim 4, wherein the laser beam is focused to a predetermined position and said optoacoustic signal is overlaid over a real-time 2D ultrasound image so as to establish a target for treatment and treatment monitoring of the tissue at the predetermined target position.

7. The apparatus as claimed in claim 5, wherein the laser beam is focused to a predetermined position and said optoacoustic signal is overlaid over a real-time 2D ultrasound image so as to establish a target for treatment and treatment monitoring of the tissue at the predetermined target position.

8. The apparatus as claimed in claim 6, wherein the predetermined position is a ciliary body in the eye.

9. The apparatus as claimed in claim 7, wherein the predetermined position is a ciliary body in the eye.

10. The apparatus as claimed in claim 1, wherein said radiation imparts power for treatment.

11. The apparatus as claimed in claim 4, wherein a standoff is provided to said probe.

12. The apparatus as claimed in claim 5, wherein a standoff is provided to said ultrasound probe.

13. An ultrasound imaging apparatus supporting pulse-echo ultrasound modes of operation as well as optoacoustic, (thermoacoustic) ultrasound mode of operation and displaying simultaneously the mode relevant images overlaid one on top of the other on a combined image; the system comprising:
conventional pulse-echo ultrasound probe;
pulsed electro-magnetic source generating radiation;
control lines connecting said pulsed electro-magnetic source and the ultrasound imaging apparatus.

14. The apparatus as claimed in claim 13, wherein said source is selected from a group of radiation sources such as laser, microwave, or radio frequency.

15. The apparatus as claimed in claim 13, wherein the apparatus is adapted to perform measurement of the concentration of substances in body fluids and generating an optoacoustic image combined with a pulse-echo ultrasound image, as a function of said concentration.

16. The apparatus as claimed in claim 13, further comprising an attachment fixed to the conventional pulse-echo ultrasound probe that includes an optical fiber allowing a laser beam to be directed to a predetermined position relative to said ultrasound probe, wherein said laser beam generates radiation that is adapted to be directed to the predetermined position.

17. The apparatus as claimed in claim 16, wherein the laser beam is focused to the predetermined position and the optoacoustic signal is overlaid over a real-time 2D ultrasound image so as to establish a target for treatment and treatment monitoring of the tissue at the predetermined target position.

18. The apparatus as claimed in claim 13, wherein said radiation imparts power for treatment.

19. The apparatus as claimed in claim 13, wherein said radiation is delivered to the body surface through an optical fiber that is an integral part of said pulse-echo ultrasound probe.

20. The apparatus as claimed in claim 16, wherein the predetermined position is a ciliary body in the eye.

21. The apparatus as claimed in claim 13, wherein the operation sequence comprising:
performing a first sequence of at least 1 pulse-echo along a predetermined axial direction;
performing a second sequence of at least one electromagnetic excitation along said axial direction;
performing said first sequence and said second sequence along multitude axial directions;
constructing a final image from signals received from said multitude axial directions.

22. The apparatus as claimed in claim 13, wherein the operation sequence comprises:
generating one complete pulse-echo image frame;
generation a complete electromagnetically excited image frame to produce a final image; and
combining final image from said pulse-echo images frame and excited image frame.
